(19) United States
(12) Patent Application Publication
(10) Pub. No.: US 2015/0238444 A1
(43) Pub. Date: Aug. 27, 2015

(54) CHLORHEXIDINE GLUCONATE SOLUBILIZED IN A HYDROPHOBIC MONOACYLGLYCERIDE

(71) Applicant: 3M INNOVATIVE PROPERTIES COMPANY, St. Paul, MN (US)

(72) Inventors: Vinod P. Menon, Woodbury, MN (US); Joseph D. Rule, Woodbury, MN (US); Richard B. Ross, Cottage Grove, MN (US)

(21) Appl. No.: 14/424,198

(22) PCT Filed: Aug. 27, 2013
(86) PCT No.: PCT/US2013/056802

§ 371 Date: Feb. 26, 2015

Related U.S. Application Data
(60) Provisional application No. 61/694,080, filed on Aug. 28, 2012.

(51) Int. Cl. A61K 31/155; A61K 47/14

(52) U.S. Cl. CPC A61K 31/155; A61K 47/14

(57) ABSTRACT

Chlorhexidine gluconate solubilized in a hydrophobic monoacylglyceride is described. Compositions incorporating such materials, as well as methods of preparing such materials are also described.

### FIELD

The present disclosure relates to chlorhexidine gluconate solubilized in a hydrophobic monoacylglyceride. The present disclosure also relates to compositions incorporating such material, as well as methods of preparing such materials.

### SUMMARY

Briefly, in one aspect, the present disclosure provides a composition comprising chlorhexidine gluconate solubilized in a monoacylglyceride having a hydrophilic-lipophilic balance of no greater than 10.

In some embodiments, the monoacylglyceride has a solubility parameter of greater than 10 (cal/cm³)^{1/2} In some embodiments, the monoacylglyceride has a binding energy to chlorhexidine gluconate of at least 25 kilocalories per mole. In some embodiments, the monoacylglyceride comprises vicinal hydrogen-bonding groups.

In some embodiments, the acyl group of the monoacylglyceride is a C8 to C18 acyl group, e.g., glycerol monocaprylate, glycerol monolaurate, glycerol monoisostearate, glycerol monooleate, and combinations thereof.

In some embodiments, the composition comprises no greater than 1 part by weight monoacylglyceride per 1 part by weight chlorhexidine gluconate. In some embodiments, the composition comprises no greater than 0.1 parts by weight water per 1 part by weight chlorhexidine gluconate.

In another aspect, the present disclosure provides the composition further comprises a carrier. In some embodiments, the carrier comprises one or more of a hydrophobic vehicle other than a monoacylglyceride, a polymer, and a solvent. In some embodiments, the composition further comprises a thixotropic agent.

The above summary of the present disclosure is not intended to describe each embodiment of the present invention. The details of one or more embodiments of the invention are also set forth in the description below. Other features, objects, and advantages of the invention will be apparent from the description and from the claims.

### DETAILED DESCRIPTION

Chlorhexidine digluconate, commonly referred to as “chlorhexidine gluconate” or “CHG,” is an antimicrobial useful in various applications. CHG is often provided as an aqueous solution, in part because CHG may degrade in a non-aqueous composition. CHG has been provided in non-aqueous solutions by replacing water with a hydrophilic vehicle. For example, U.S. Pat. No. 6,458,341 (Rozzi et al., issued Oct. 1, 2002) describes non-aqueous solutions containing CHG and a solubilizing glycol.

Surprisingly, the present inventors have discovered that CHG can be solubilized in a hydrophobic vehicle. Consistent with typical usage, as used herein, a “hydrophobic vehicle” is one having a hydrophile/lipophile balance (“HLB”) of no greater than 10. For example, in some embodiments, the compositions comprise at least 5% by weight CHG dissolved in the hydrophobic vehicle based on the combined weight of the CHG and hydrophobic vehicle. In some embodiments, the compositions comprise at least 10%, e.g., at least 15%, or even at least 20% by weight CHG dissolved in the hydrophobic vehicle based on the combined weight of the CHG and hydrophobic vehicle.

Three distinct methods are available for preparing non-aqueous solutions of CHG in hydrophobic vehicles. The first method involves lyophilizing CHG, and then dissolving the CHG into the hydrophobic vehicle. The second method involves mixing an aqueous CHG solution with a relatively high boiling hydrophobic vehicle, and then pulling a vacuum on the mixture to remove the water. The third method involves generating the CHG in situ by reacting gluconolactone, a limited amount of water, and chlorhexidine free base. Each method is expected to give a similar final product, i.e., CHG dissolved in a hydrophobic vehicle, but each method has advantages and disadvantages. For example, the lyophilization route does not require exposing the CHG to sustained heat, which helps prevent degradation. The liquid vacuum stripping route is easily scalable using readily available manufacturing equipment, e.g., kettles. The in situ generation method does not require vacuum-equipped reactors. All the methods may leave small amounts of water behind. Therefore, as used herein, “non-aqueous” refers to compositions that may contain small amounts of water, e.g., less than 5 wt. %, based on the total weight of the composition. In some embodiments, the compositions contain less than 2 wt. %, e.g., less than 1 wt. %, or even less than 0.1 wt. % water, based on the total weight of the composition.

In some embodiments, the compositions contain little or no hydrophilic vehicle, i.e., vehicles having an HLB of greater than 10. As used herein, water is considered a separate component independent of any hydrophilic vehicles; therefore, the following amounts are exclusive of any water which may be present in the composition. In some embodiments, the compositions comprise no greater than 2 parts by weight hydrophilic vehicle per 1 part by weight CHG, e.g., no greater than 1 part by weight, no greater than 0.5 part by weight, or even no greater than 0.1 parts by weight hydrophilic vehicle per 1 part by weight CHG.

In some embodiments, the compositions comprise no greater than 1 part by weight water per 1 part by weight CHG, e.g., no greater than 0.5 part by weight, no greater than 0.1 part by weight, or even no greater than 0.01 parts by weight water per 1 part by weight CHG.

Hydrophobic vehicles have an HLB value of no greater than 10. In some embodiments, the hydrophobic vehicle has an HLB value of no greater than 9, e.g., no greater than 7.

Generally, the hydrophobic vehicle is a monoacylglyceride, e.g., 1-monoacylglycerides having the formula:

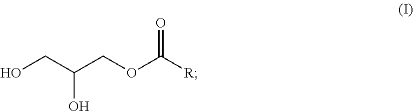

and 2-monoacylglycerides having the formula:

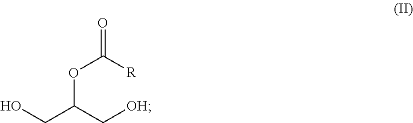

wherein RC═O is the acyl group. Such monoacylglycerides include proximate hydroxyl groups. As used herein, “proximate” groups refer to groups separated by no more than three carbon atoms, e.g., the 2-monoacylglycerides of Formula II. In some embodiments, the proximate groups may be vicinal, i.e., separated by two carbon atoms, e.g., the 1-monoacylglycerides of Formula I.

In some embodiments, the acyl group of the monoacylglyceride is a C8 to C18 acyl group. In some embodiments, the R-group of the acyl group is linear. In some embodiments, the R-group is branched. In some embodiments, the R-group is saturated. In some embodiments, the R-group is unsaturated. Often monoacylglycerides are available as a mixture containing a range of acyl group chain lengths. Exemplary monoacylglycerides include, e.g., glycerol monocaprylate, glycerol monolaurate, glycerol monoisostearate, glycerol monooleate, and combinations thereof. Often such materials are commercially available as mixtures containing both the 1-monoacylglyceride and the 2-monoacylglyceride.

Examples.

Objects and advantages of various embodiments of the present disclosure are further illustrated by the following examples, but the particular materials and amounts thereof recited in these examples, as well as other conditions and details, should not be construed to unduly limit this invention. Unless otherwise indicated, all parts and percentages are on a weight basis, all water is distilled water, and all molecular weights are weight average molecular weight.

General Method for Vacuum-Stripping Water from CHG Solutions. A 25 g sample of CHG/water solution (20 wt. CHG) was added to 45 g of the hydrophobic monoacylglyceride of interest in a 200 mL round bottom flask. The flask was put in a 60° C. oil bath and stirred with a magnetic stir bar. Vacuum (less than 3.3 kilopascal (25 Torr)) was pulled until bubbling stopped and the weight had closely approached the theoretical value of 50 grams—usually 30-90 minutes. In some cases, the hydrophobic monoacylglyceride was somewhat volatile and the weight of the solution decreased significantly below 50 grams. In those cases, after nearly all the water was removed, additional hydrophobic monoacylglyceride was added to bring the final weight to 50 grams.

Screening tests were conducted to determine the solubility of CHG in a variety of hydrophobic vehicles. Samples were prepared by mixing CHG (20 wt. % solution in water, obtained from Xttrium Laboratories Inc., Chicago, Ill.) with the vehicle and vacuum stripping in accordance with the General Method for Vacuum-Stripping Water from CHG Solutions. The resulting concentration of CHG and residual water were determined. Also, the final state of the solution was qualitatively evaluated to determine whether the mixture was transparent and apparently homogenous, or clearly inhomogeneous. The results obtained using compounds with ester groups are summarized in Tables 1 and 2.

TABLE 1
Vehicles with ester groups providing good CHG solubility.

| | Alkyl | CHG. | Water | Temper- | | |
|---|---|---|---|---|---|---|
| Compound | group | wt. % | wt. % | ature | | Final State |
| Glyceryl | C8 | 20% | ND | 23-60° | C. | Homogeneous |
| monocaprylate | | | | | | |
| Glyceryl | C12 | 20% | 0.7% | 75° | C. | Homogeneous |
| monolaurate | | | | | | |
| Glyceryl | C18 | 10% | 0.3% | 23-60° | C. | Homogeneous |
| monooleate | | | | | | |
| Glyceryl | C18 | 10% | 0.2% | 80° | C. | Homogeneous |
| monostearate | | | | | | |
| Glyceryl | C18 | 20% | ND | 60° | C. | Homogeneous |
| monoisostearate | | 16% | 0.1% | 23-60° | C. | Homogeneous |
| | | 10% | 0.7% | 23-60° | C. | Homogeneous |
| Medium Chain | mix | 20% | 0.3% | 23-50° | C. | Homogeneous |
| Monoglyceride | | | | | | |

ND = not determined

TABLE 2
Vehicles with ester groups providing poor CHG solubility.

| | CHG | | |
|---|---|---|---|
| Compound | wt. % | Temperature | Final State |
| Dialkyl (C12 & C13) Tartrate | 10% | 23-50° C. | Inhomogeneous |
| Trigyceryl diisostearate | 10% | 23-60° C. | Inhomogeneous |
| | 1% | 23-60° C. | Inhomogeneous |
| Polyglycerol-2-Triisostearate | 10% | 23-60° C. | Inhomogeneous |

Molecular dynamics simulations were used to investigate underlying causes of observed differences in solubilities with CHG. Simulations were independently carried out to find optimal binding structures of several of the hydrophobic vehicles with CHG. Solubility parameters were also been computed via molecular dynamics simulations for several of the vehicles.

HLB values were calculated using the method of Griffin (Griffin W C; J. Soc. of Cosmetic Chemists 5, 259 (1954)) (the “HLB Method”). In this method,

HLB=(E+P)/5,

where E is the weight percent of oxyethylene content and P is the weight percent of polyhydric alcohol content (glycerol, sorbitol, etc). For the compounds herein, glycerol segments with two hydroxyl groups, glycerol segments with one hydroxyl group, and hydroxyl-containing segments of any additional polyhydric molecules were included in the definition of P.

Other methods of calculating HLB are available and may be required when determining the HLB value for compounds lacking both E and P groups, as defined above. While the calculated value of HLB may vary depending on the method used, the trends and relative hydrophobicity of materials are expected to be similar.

The Solubility Parameter Method. Solubility parameters were computed via molecular dynamics simulations using the general procedures as described by Belmares et al. (Belmares, M.; Blanco, M.; Goddard, W. A.; Ross, R. B.; Caldwell, G; Chou, S. H.; Pham, J.; Olofson, P. M.; Thomas, C.; J. Comp. Chem., 25 (15), 1814 (2004), as implemented in Culgi software (Culgi Software, Culgi BV, P.O. Box 252, 2300 AG Leiden, The Netherlands).

The Binding Energy Method. Binding energies were computed using quench molecular dynamics simulations employing QEq charges (Rappe, A. K.; Goddard, W. A.; J. Phys. Chem. 95, 3358 (1991)) and the Dreiding Force Field (Mayo, S. L., Olafson, B. D., Goddard, W. A.; J. Phys. Chem. 94, 8897 (1990)) as implemented in Materials Studio Software (Materials Studio, Accelrys, Software Inc., 10188 Telesis Court, Suite 100, San Diego, Calif. 92121). Multiple independent simulations were carried out for each vehicle/CHG pair. Typically, 100,000 steps of simulation at 298K were carried out between the hydrophobic vehicle of interest and CHG. The simulation was typically “quenched” or minimized into an optimized complex structure every 5000 steps. Based on the results of independent simulations, the binding energy for the most optimized (lowest energy) complex structure was found using the equation:

$$Binding Energy = \frac{E ( HV ) + E ( CHG ) - E ( HV * CHG )}{HBN}$$

wherein E(HV) is the energy of the hydrophobic vehicle, E(CHG) is the energy of the CHG, and E(CHG*HV) is the energy of the CHG*hydrophobic vehicle complex. The Hydrogen Bonding Number (HBN) is a normalization factor to account for varied numbers of hydrogen bonding groups in the hydrophobic vehicles. HBN was computed as a weighted sum of the number of hydrogen-bonding donor/acceptor groups and hydrogen-bonding acceptor-only groups. The hydrogen-bonding donor/acceptor groups (e.g., OH) were given a weighting of 1. The hydrogen-bonding acceptor-only groups (e.g., O in C═O or O in C—O—C) were given a weighting of ½. This general procedure was similar to that used by Zhang et al. to compute binding energies between polymers and hydroxy apatite (Zhang, H. P.; Xiong, L; Leng; Y, Fang, L.; Qu, Shuxin; Feng, B.; Weng, J.; Wang, J.; Acta Biomaterialia 5(4), 1169 (2009)). In the present application, the binding energies were computed between organic compounds as opposed to between an organic and inorganic compound as in the Zhang et al. studies.

A summary of calculated HLB, solubility parameter, and binding energy for various hydrophobic vehicles with CHG are shown in Table 3, along with CHG experimental solubility data for comparison. As can be seen in this table, vehicles with a computed solubility parameter greater than 10 were experimentally found to provide CHG-solubility, while those with a computed solubility parameter of less than 10 did not. In addition, vehicles that were experimentally found to provide CHG-solubility had computed CHG binding energies of greater than 25 kcal/mol.

TABLE 3
Calculated Solubility Parameter, Normalized Binding Energy,
and CHG-solubility for a variety of hydrophobic vehicles.

| | | Solubility | Binding | Experimental |
|---|---|---|---|---|
| | | Parameter | Energy | Solubility |
| Hydrophobic Vehicle | HLB | (cal/cm³)^{1/2} | (kcal/mol) | with CHG |
| Glyceryl monocaprylate | 8.4 | 13.1 | 27 | Yes |
| Glyceryl monolaurate | 6.6 | 13.9 | 29 | Yes |
| Glycerol monooleate | 5.1 | 10.8 | 28 | Yes |
| Glyceryl monostearate | 5.1 | 10.8 | 32 | Yes |
| Glyceryl monoisostearate | 5.1 | 10.8 | 28 | Yes |
| Dialkyl (C12 & C13) | 4.2 | 9.6 | 21 | No |
| Tartrate | | | | |
| Trigyceryl diisostearate | 5.8 | 9.6 | 14 | No |
| Polyglycerol-2- | 3.4 | 8.5 | 32 | No |
| Triisostearate | | | | |

Analysis of optimized molecular simulation binding structures indicated that sterically unhindered vehicles with proximate hydrogen bonding groups can form exceptionally high binding complexes with CHG due to multiple hydrogen bonds between CHG and the vehicle. The proximate hydrogen bonding groups in the vehicle act in a synergistic binding manner to enable the highly hydrogen-bound complexes much like the mechanical action of a zipper in which zipping one-link enables easier zipping of the next link. Separating or adding steric hindrance to regions near the proximate hydrogen bonding groups can prevent this synergy.

In some embodiments, the compositions may include a carrier in addition to the monoacylglyceride vehicle. In some embodiments, the carrier may comprise another hydrophobic vehicle. In some embodiments, the carrier may comprise a polymer. In some embodiments, the carrier may comprise a solvent. In some embodiments, the carrier may comprise a thixotropic agent, i.e., an agent which renders the composition shear thinning.

Various modifications and alterations of this invention will become apparent to those skilled in the art without departing from the scope and spirit of this invention.

1. A composition comprising chlorhexidine gluconate solubilized in a monoacylglyceride, wherein the monoacylglyceride is hydrophobic monoacylglyceride having a hydrophilic-lipophilic balance of no greater than 10 as determined using the HLB Method.

2. The composition of claim 1, comprising at least 5% by weight chlorhexidine gluconate dissolved in the hydrophobic monoacylglyceride based on the combined weight of the CHG and hydrophobic monoacylglyceride.

3. The composition of claim 1, wherein the monoacylglyceride has a solubility parameter of greater than 10 (cal/cm³)^{1/2}.

4. The composition according to claim 1, wherein the monoacylglyceride has a binding energy to chlorhexidine gluconate of at least 25 kilocalories per mole.

5. The composition according to claim 1, wherein the monoacylglyceride comprises two vicinal hydrogen-bonding groups.

6. The composition according to claim 1, wherein the acyl group of the monoacylglyceride is a C8 to C18 acyl group.

7. The composition of claim 6, wherein the monoacylglyceride is selected from the group consisting of glycerol monocaprylate, glycerol monolaurate, glycerol monoisostearate, glycerol monooleate, and combinations thereof.

8. The composition according to claim 1, wherein the composition comprises no greater than 1 part by weight hydrophilic vehicle per 1 part by weight chlorhexidine gluconate.

9. The composition according to claim 1, wherein the composition comprises no greater than 0.1 parts by weight hydrophilic vehicle per 1 part by weight chlorhexidine gluconate.

10. The composition according to claim 1, wherein the composition comprises no greater than 0.1 parts by weight water per 1 part by weight chlorhexidine gluconate.

11. The composition according to claim 1, further comprising a carrier.

12. The composition of claim 11, wherein the carrier comprises a polymer.

13. The composition of claim 11, wherein the carrier comprises an organic solvent.

14. The composition according to claim 1, further comprising a thixotropic agent.
